(19) United States
(12) Patent Application Publication
(10) Pub. No.: US 2019/0200898 A1
(43) Pub. Date: Jul. 4, 2019

(54) COMPUTER, METHOD FOR ACQUIRING RESPIRATION RATE, AND INFORMATION PROCESSING SYSTEM

(71) Applicants: Sharp Kabushiki Kaisha, Sakai City, Osaka (JP); Osaka Prefecture University Public Corporation, Sakai-shi, Osaka (JP)

(72) Inventors: Hiroshi SAKAYA, Sakai City (JP); Tetsuya HAYASHI, Sakai City (JP); Azusa NAKANO, Sakai City (JP); Syunsuke SHIMAMURA, Sakai-shi (JP); Terumasa SHIMADA, Sakai-shi (JP)

(21) Appl. No.: 16/332,805

(22) PCT Filed: Aug. 29, 2017
(86) PCT No.: PCT/JP2017/030968

§ 371 Date: Mar. 13, 2019

(30) Foreign Application Priority Data
Sep. 20, 2016 (JP) 2016-182683

(51) Int. Cl. A61B 5/08; A61B 5/0245; A01K 29/00; A61B 5/00; A61B 5/024

(52) U.S. Cl. CPC A61B 5/0816; A61B 5/0245; A61B 5/02438; A61B 5/0022; A01K 29/005

(57) ABSTRACT

Computers are that include interfaces configured to acquire data indicating a pulse or heartbeat of an animal, and processors configured to determine whether a prescribed condition is satisfied or not based on the data indicating the pulse or the heartbeat of the animal, and calculate a respiratory rate in a period of time for which the prescribed condition is satisfied, are provided.

### TECHNICAL FIELD

This application claims the benefit of priority to JP 2016-182683 filed on Sep. 20, 2016. The entire contents of the above-identified application are hereby incorporated by reference.

The following disclosure relates to a technique for acquiring a respiratory rate of an animal.

### BACKGROUND ART

Conventionally, techniques for acquiring a respiratory rate of an animal have been known. For example, JP 62-22627 A (PTL 1) discloses a respiratory rate measuring device. According to PTL 1, a pulse interval is detected, a change cycle of the pulse interval is detected, and a respiratory rate in unit time is calculated from an inverse of the change cycle.

Further, J P 2014-133049 A (PTL 2) discloses a vital information management module, a sleep meter, and a control device. According to PTL 2, the vital information management module includes a first acquisition unit, a determination unit, and a generation unit. The first acquisition unit acquires a plurality of different kinds of vital information during sleep as a vital information group. The determination unit determines a state of an animal based on the vital information group. The generation unit generates an execution command when the determination unit determines that the state of the animal is a prescribed state. The execution command causes a first device to execute a prescribed operation. The first device executes the prescribed operation for the animal.

### CITATION LIST

### Patent Literature

PTL1: JP 62-22627 A
PTL2: JP 2014-133049 A

### SUMMARY OF INVENTION

### Technical Problem

A technique capable of acquiring a respiratory rate of an animal per unit time more efficiently than those in the related art is in demand. An embodiment of the present invention is to solve the problem, and an objective of the present invention is to provide a computer, a respiratory rate acquisition method, and an information processing system capable of acquiring a respiratory rate of an animal per unit time more efficiently than in the past.

### Solution to Problem

According to an embodiment of the present invention, a computer that includes an interface configured to acquire data indicating a pulse or heartbeat of an animal, and a processor configured to determine whether a prescribed condition is satisfied or not based on the data indicating a pulse or heartbeat of an animal, and calculate a respiratory rate in a period of time during which a prescribed condition is satisfied, is provided.

The processor is preferably configured to calculate a respiratory rate from the data of a pulse or heartbeat of an animal.

The processor is preferably configured to process the data of a pulse or heartbeat of an animal sequentially, and to calculate a respiratory rate from the data of a pulse or heartbeat of an animal in a period of time during which a prescribed condition is satisfied.

The processor is preferably configured to calculate an inter-beat interval from data of a pulse or heartbeat of an animal, and calculate a respiratory rate based on an inter-beat interval.

The processor is preferably configured to create a power spectrum of an inter-beat interval, to determine whether a prescribed condition is satisfied or not based on the power spectrum, and acquire a respiratory rate based on the power spectrum.

The processor is preferably configured to determine whether a prescribed condition is satisfied or not based on a Poincaré plot of an inter-beat interval.

According to another aspect of the present invention, a method of acquiring a respiratory rate of an animal, the method performed on a computer including a processor, is provided. The acquisition method includes a step for acquiring data indicating a pulse or heartbeat of an animal, a step for determining whether a prescribed condition is satisfied or not based on data indicating a pulse or heartbeat of an animal, and a step for acquiring a respiratory rate in a period of time during which a prescribed condition is satisfied.

According to another aspect of the present invention, an information processing system is provided, the information processing system including an output device, a sensor configured to detect a beat of an animal, and a computer, the computer being configured to determine whether a prescribed condition is satisfied or not based on data indicating a pulse or heartbeat of an animal from a sensor, to calculate a respiratory rate in a period of time during which the prescribed condition is satisfied, and to cause the output device to output.

### Advantage Effects of Invention

As described above, according to an embodiment of the present invention, a computer, a respiratory rate acquisition method, and an information processing system capable of acquiring a respiratory rate of an animal per unit time more efficiently than those in the related art are provided.

### BRIEF DESCRIPTION OF DRAWINGS

FIG. 1 is an example of an overall configuration of an information processing system 1 according to a first embodiment.

FIG. 2 is a diagram illustrating a functional configuration of the information processing system 1 according to the first embodiment.

FIG. 3 is a diagram illustrating a hardware configuration of a signal processing apparatus 500 according to the first embodiment.

FIG. 4 is a flowchart illustrating a processing procedure of the information processing system 1 according to the first embodiment.

FIG. 5 is an example of electrocardiac data and inter-beat intervals according to the first embodiment.

FIG. 6 is an example of relation between beat detection timing and an inter-beat interval according to the first embodiment.

FIG. 7 is an example of power spectral distribution according to the first embodiment.

FIG. 8A is an example of RRI fluctuation after spline interpolation, and FIG. 8B is an example of power spectral distribution, of a dog at rest according to the first embodiment.

FIG. 9A is an example of RRI fluctuation after spline interpolation, and FIG. 9B is an example of power spectral distribution, of a dog on excitation according to the first embodiment.

FIGS. 10A and 10B are an example of an effect of a respiratory rate acquisition method according to the first embodiment.

FIG. 11 is a flowchart illustrating a processing procedure of the information processing system 1 according to a third embodiment.

FIG. 12 is an example of a correspondence relation table between an inter-beat interval R−R(n) and a next inter-beat interval R−R(n+1) according to the third embodiment.

FIG. 13 is an example of a conversion from a correspondence relation table 321A between an inter-beat interval R−R(n) and a next inter-beat interval R−R(n+1) to an axis in a Y=X direction and an axis in a direction perpendicular to the Y=X direction according to the third embodiment.

FIG. 14 is a table showing standards of standard deviations with respect to the Y=X axis, and standard deviations with respect to a Y=−X axis, per states of a dog according the third embodiment.

FIG. 15 is an example of a Poincaré plot of a dog in a resting state according to the third embodiment.

FIG. 16 is an example of a Poincaré plot of a dog in an excited state according to the third embodiment.

FIG. 17 is an example of time series change in inter-beat intervals according to the third embodiment.

FIG. 18 is a diagram illustrating a functional configuration of the information processing system 1 according to a fourth embodiment.

FIG. 19 is a flowchart illustrating a processing procedure of the information processing system 1 according to the fourth embodiment.

FIG. 20 is an example of an overall configuration of the information processing system 1 according to a fifth embodiment.

FIG. 21 is an example of an overall configuration of the information processing system 1 according to a sixth embodiment.

FIG. 22 is an example of an overall configuration of the information processing system 1 according to a seventh embodiment.

FIG. 23 is a diagram illustrating a functional configuration of the information processing system 1 according to the seventh embodiment.

FIG. 24 is an example of an overall configuration of the information processing system 1 according to an eighth embodiment.

FIG. 25 is an example of an overall configuration of the information processing system 1 according to a ninth embodiment.

FIG. 26 is a diagram illustrating a functional configuration of the information processing system 1 according to the ninth embodiment.

FIG. 27 is a diagram illustrating a hardware configuration of a communication terminal 300F according to the ninth embodiment.

FIG. 28 is an example of an overall configuration of the information processing system 1 according to a tenth embodiment.

FIG. 29 is a diagram illustrating a functional configuration of the information processing system 1 according to the tenth embodiment.

FIG. 30 is a diagram illustrating a hardware configuration of a server 100G according to the tenth embodiment.

FIG. 31 is an example of another overall configuration of the information processing system 1 according to the tenth embodiment.

FIG. 32 is a Poincaré plot diagram of a dog in an excited state according to the third embodiment.

FIG. 33 is a Poincaré plot diagram of a dog with steady breathing in a normal state according to the third embodiment.

FIG. 34 is a Poincaré plot diagram of a dog in a normal state according to the third embodiment.

FIG. 35 is a Poincaré plot diagram of a dog in a resting state according to the third embodiment.

FIG. 36 is a diagram made by plotting respiratory rates per minute for four subjects to be tested measured in each of a first experimental example and a first comparative example.

### DESCRIPTION OF EMBODIMENTS

Embodiments of the present invention will be described below with reference to the drawings. In the following descriptions, identical components are given identical signs. Respective names and functions of the components are also identical. Thus, detailed descriptions will not be repeated for the components.

### First Embodiment

### Overall Configuration of Information Processing System

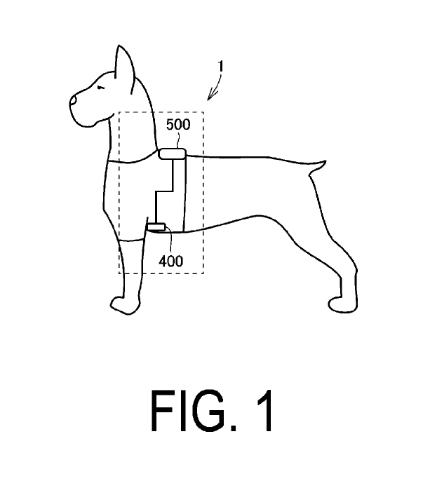

First, with reference to FIG. 1, an overall configuration of the information processing system 1 will be described. FIG. 1 is an example of an overall configuration of the information processing system 1 according to the present embodiment.

The information processing system 1 mainly includes, an electrode 400 attached to a chest of an animal and configured to acquire an electrocardiographic signal, and the signal processing apparatus 500 configured to process the electrocardiographic signal to calculate a respiratory rate. The information processing system 1 is configured such that a vest-shaped measuring device is attached on a subject to be tested such as a dog, the respective electrodes 400 are attached on left and right axillary portions of an animal, and the signal processing apparatus 500 and the like are provided on a dorsal side. Note that a configuration of a device is not limited to this.

### Functional Configuration and Processing Procedure of Information Processing System

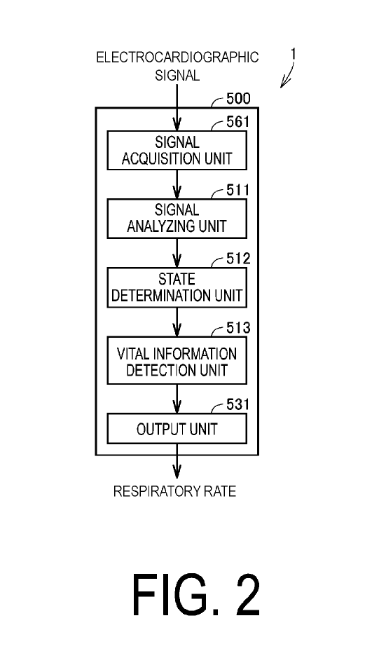

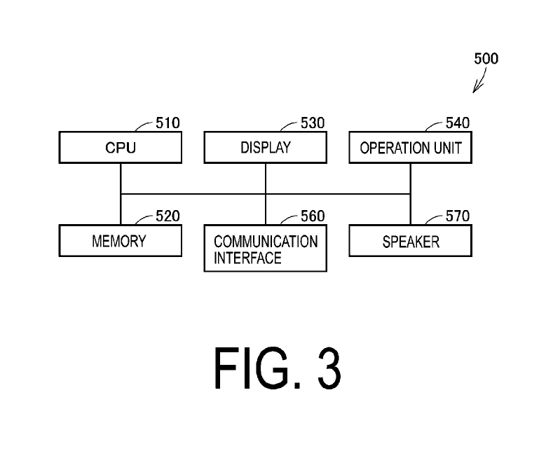

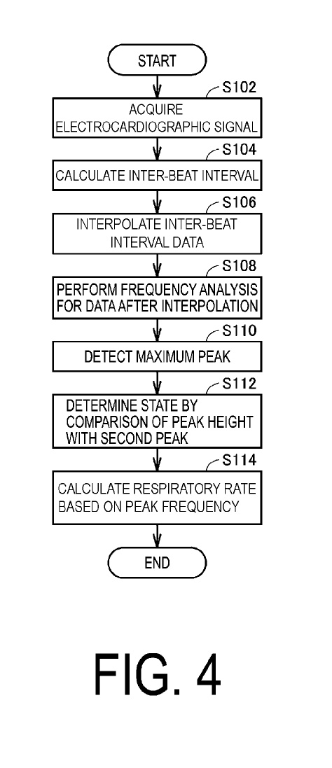

Next, with reference to FIG. 2 to FIG. 4, a configuration and a processing procedure of the information processing system 1 according to the present embodiment will be described. FIG. 2 is a diagram illustrating a functional configuration of the information processing system 1 according to the present embodiment. FIG. 3 is an example of a hardware configuration of the signal processing apparatus 500 according to the present embodiment. FIG. 4 is a flowchart illustrating a processing procedure of the information processing system 1 according to the present embodiment.

First, the signal processing apparatus 500 of the information processing system 1 includes a signal acquisition unit 561, a signal analyzing unit 511, a state determination unit 512, a vital information detection unit 513, and an output unit 531.

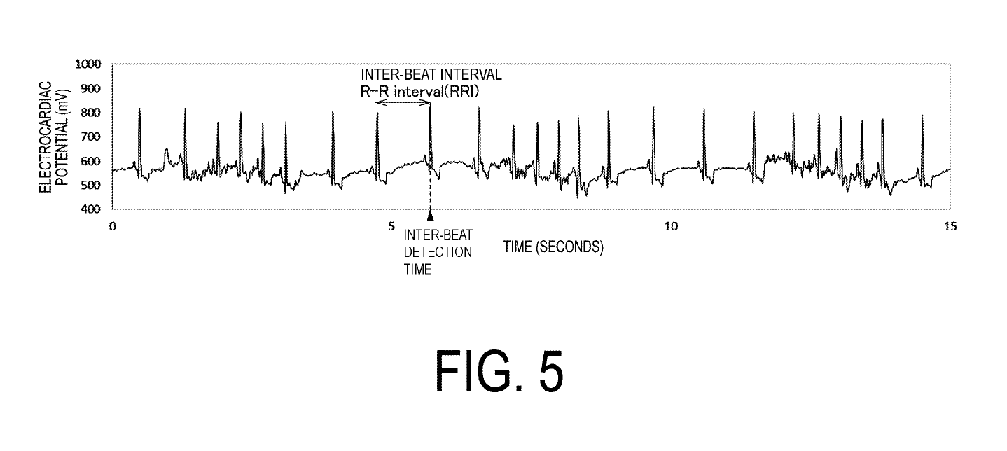

The signal acquisition unit 561 includes an electrocardiograph, a communication interface 560, a filter, an amplifier, and the like. The signal acquisition unit 561, as illustrated in FIG. 5, for example, sequentially acquires an electrocardiographic signal at 100 Hz, and hands over the electrocardiographic signal to the signal analyzing unit 511 (step S102).

A central processing unit (CPU) 510 executes programs stored in a memory 520, so that the signal analyzing unit 511 is achieved. The signal analyzing unit 511 sequentially calculates beat detection time and inter-beat intervals as illustrated in FIG. 5 from the electrocardiographic signal acquired from the signal acquisition unit 561 (step S104).

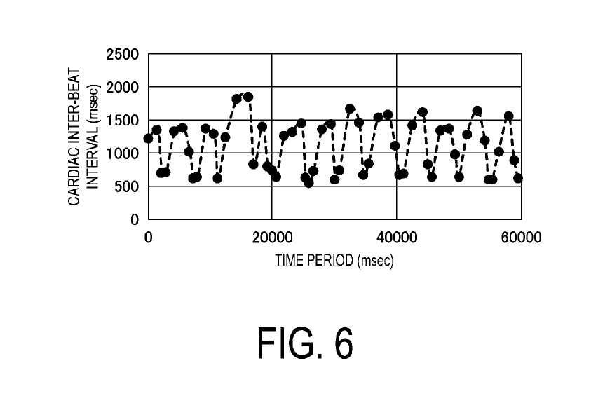

Additionally, the signal analyzing unit 511, for example, as illustrated in FIG. 6, mathematically interpolates (e.g., spline interpolation) relation between beat detection time and an inter-beat interval for one minute (step S106). More specifically, the signal analyzing unit 511 detects a peak signal (R wave) among electrocardiographic signals by a method such as threshold detection, and calculates respective periods (times) between the peaks of the electrocardiographic signals. As a calculation method of the inter-beat interval, in addition to the above method, derivation of a cycle using an autocorrelation function, a method using a square wave correlation trigger, or the like, may be adopted.

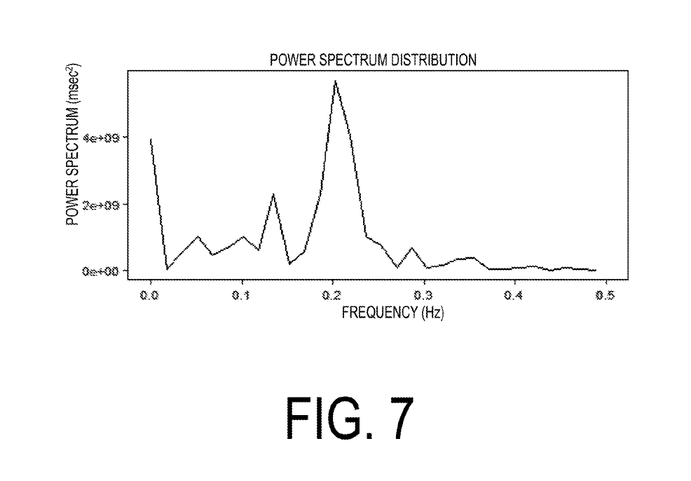

Additionally, the signal analyzing unit 511, as illustrated in FIG. 7, performs frequency analysis by an acquired function (step S108).

The CPU 510 executes the programs stored in the memory 520, so that the state determination unit 512 is achieved. The state determination unit 512, in power spectral distribution as in FIG. 7 acquired by the frequency analysis in the signal analyzing unit 511, and within an arbitrary frequency range (e.g., from 0.05 to 0.5 Hz), finds out a maximum peak of power spectrum (step S110). The state determination unit 512, when a ratio of the maximum peak compared to a second largest peak is equal to or larger than an arbitrary threshold value (e.g., three times), determines the state as a “measurable state”.

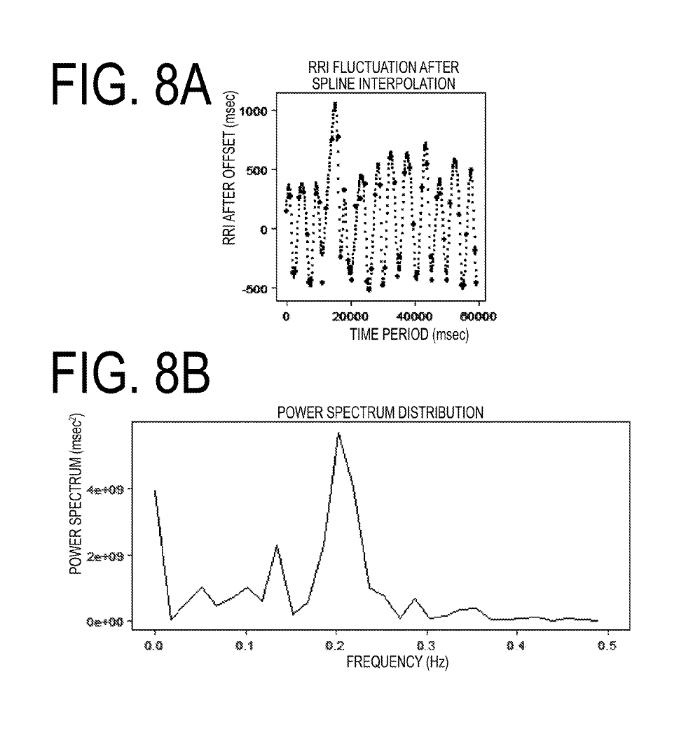

More specifically, for example, RRI fluctuation after spline interpolation of a dog in a relaxed state in a calm indoor room is as illustrated in FIG. 8A. Power spectral distribution in this case is as illustrated in FIG. 8B, and since a ratio of a maximum peak compared to a second largest peak is equal to or larger than an arbitrary threshold value (e.g., three times), the state determination unit 512 determines the state as the “measurable state”.

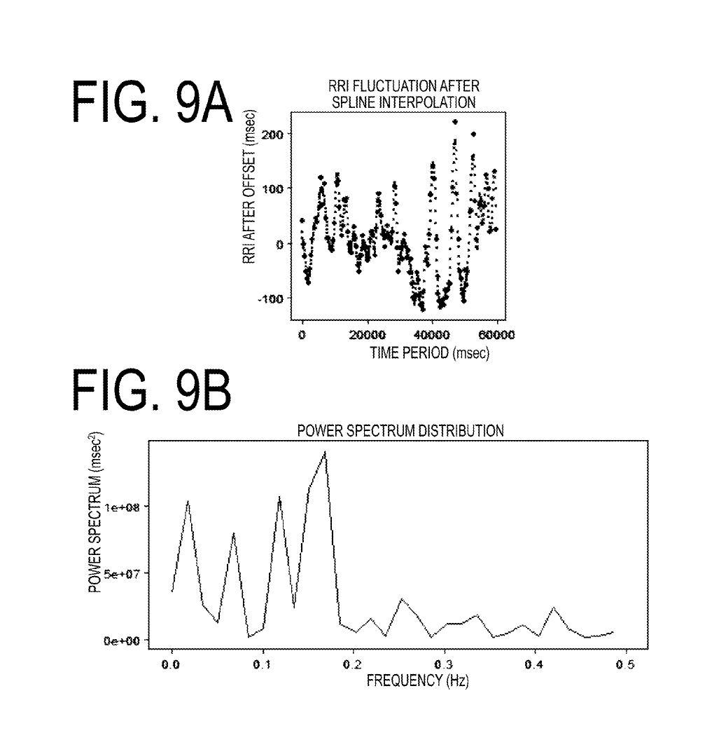

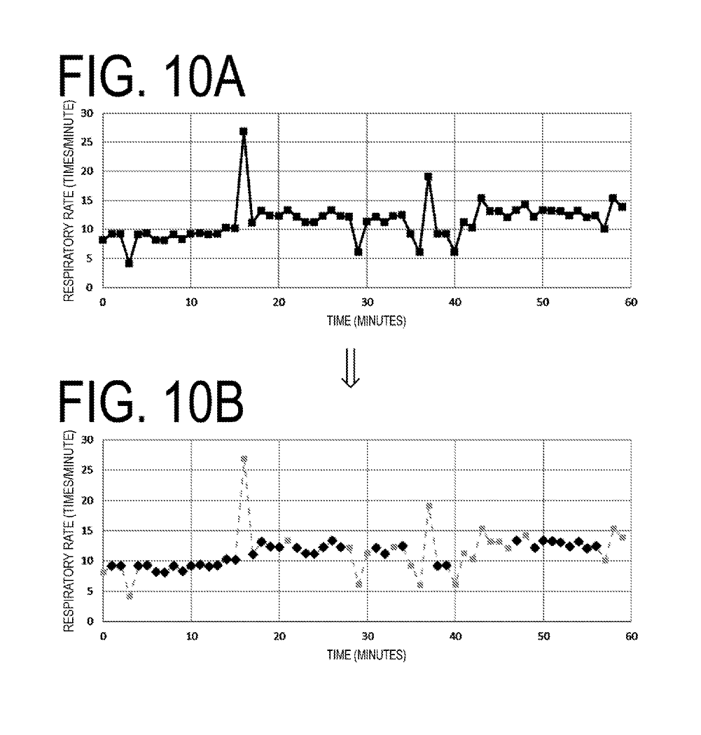

Conversely, for example, RRI fluctuation after the spline interpolation of a dog in a restless state under a noisy outdoor environment is illustrated in FIG. 9A. Power spectral distribution in this case is as illustrated in FIG. 9B, since a ratio of a maximum peak compared to a second largest peak is not equal to or larger than an arbitrary threshold value (e.g., three times), the state determination unit 512 determines the state as an “unmeasurable state”.

When the state determination unit 512 determines the state as the “unmeasurable state”, processes in the step S106 and subsequent steps are repeated based on inter-beat intervals already acquired by the signal acquisition unit 561, for another timing.

The CPU 510 executes the programs stored in the memory 520, so that the vital information detection unit 513 is achieved. The vital information detection unit 513, when the state determination unit 512 determines the state as the “measurable state”, detects vital information. The vital information detection unit 513, with a maximum peak in an arbitrary frequency range (e.g., from 0.05 to 0.5 Hz) in the frequency analysis performed in the state determination unit 512 being a breathing frequency, calculates a respiratory rate by calculating an inverse.

The output unit 531 includes a display 530, a speaker 570, a communication interface 560 configured to transmit data outward, and the like. The output unit 531 displays a respiratory rate per unit time, outputs a voice, or accumulates the respiratory rate in an external data base.

In the present embodiment, the vital information detection unit 513, with a frequency of a maximum peak in the frequency analysis performed in the state determination unit 512 being a breathing frequency, calculates a respiratory rate by calculating an inverse of the frequency. FIGS. 10A and 10B are results of respiratory rate measurement for 60 minutes. When state determination is not performed, it is possible to output a measurement result for every minute as illustrated in FIG. 10A, but measurement results under various conditions are included, and it is difficult to ensure accuracy. On the other hand, by bypassing calculation of data for time determined as in the “unmeasurable state”, it is possible to calculate respiratory rates as illustrated in FIG. 10B, and obtain only respiratory rates under appropriate conditions.

More specifically, accumulating vital data has medical significance, but it is important to compare and analyze data measured under a constant environment (e.g., at rest). In particular, in a case in which data are compared for a long term, or in a case in which a subject to be measured is not able to maintain a constant state by him/herself, it is necessary to determine a state of the subject to be measured at measurement time, in order to reliably record vital data. In particular, since a respiratory rate fluctuates voluntarily, it is difficult for the subject to be measured to consciously generate a measurable state, and a method is not established for automatically determining whether measurement is possible or not, thus far.

However, by analyzing measurement data (e.g., an electrocardiographic signal), it is possible to determine a state of a subject to be measured, and based on a result of the state determination, and calculate and record vital data (e.g., a respiratory rate derived from an electrocardiographic signal). In particular, in a method for the state determination, “whether an appropriate state is maintained or not for a certain time (e.g., one minute) during measurement” is determined. Additionally, a criterion for determining “whether an appropriate state is maintained or not” is defined from a breathing fluctuation cycle, by using heartbeat fluctuation analysis, for example. As for an animal such as a dog, heartbeat and a respiratory rate change even when no movement is observed, and based on this determination criterion, it is possible to determine an appropriate state with higher precision with the criterion for determining than a case in which movement is analyzed by using an acceleration sensor and the like. Further, by performing both the state determination and the vital data detection from single measurement data such as an electrocardiographic signal, it is possible to miniaturize and simplify a measurement device. Thus, miniaturizing a device or a system makes it possible to reduce stress or a load for the subject to be measured, and enables measurement in a more natural state.

### Second Embodiment

The first embodiment utilizes the power spectrum to determine whether a target animal is in a resting state or not. Further, in the step S110 in FIG. 4, the state determination unit 512, in the power spectral distribution as in FIG. 7 acquired by the frequency analysis in the signal analyzing unit 511, and within an arbitrary frequency range (e.g., from 0.05 to 0.5 Hz), finds out a maximum peak. In addition, the state determination unit 512, when a ratio of the maximum peak compared to a second largest peak is equal to or larger than an arbitrary threshold value (e.g., three times), determines the state as the “measurable state”.

However, as the present embodiment, in the step S110 in FIG. 4, the state determination unit 512, in the power spectral distribution acquired by the frequency analysis in the signal analyzing unit 511, and within an arbitrary frequency range (e.g., from 0.05 to 0.5 Hz), may find out a maximum peak of the power spectrum, and determine that the state is the measurable state of a respiratory rate, when a ratio in which an integrated value of the power spectrum from the peak to a half value width of the peak occupies in the whole is larger than a set threshold value.

Note that it is sufficient that the state determination unit 512 is capable of determining whether a maximum peak in an arbitrary frequency range (e.g., from 0.05 to 0.5 Hz) in power spectral distribution protrudes or not compared to other power spectra. In addition, the state determination unit 512 may determine a state as the “measurable state” by using another method.

### Third Embodiment

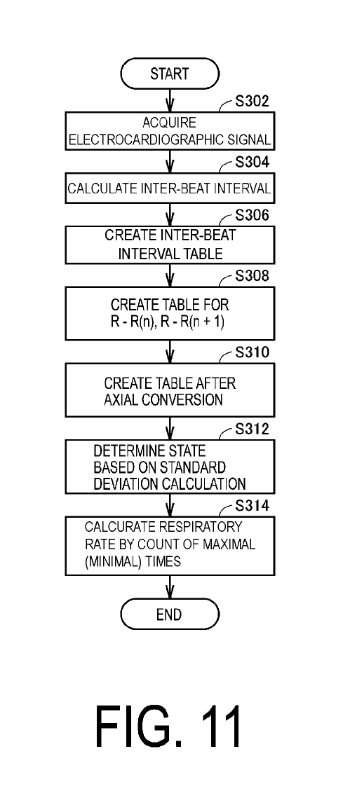

The first embodiment and the second embodiment utilize the power spectrum to determine whether the target animal is in the resting state or not. However, the signal processing apparatus 300 may determine whether the target animal is in the resting state or not, based on a Poincaré plot of inter-beat intervals. In the following, with reference to FIG. 2 and FIG. 11, a functional configuration and a processing procedure of the information processing system 1 according to the present embodiment will be described. Note that FIG. 11 is a flowchart illustrating a processing procedure of the information processing system 1 according to the present embodiment.

First, the signal acquisition unit 561, as illustrated in FIG. 5, for example, acquires an electrocardiographic signal at 100 Hz (step S302).

The signal analyzing unit 511 calculates beat detection time and an inter-beat interval as illustrated in FIG. 5 from the electrocardiographic signal acquired in the signal acquisition unit 561 (step S304). The signal analyzing unit 511 accumulates the inter-beat interval as an inter-beat interval table in the memory 520 sequentially (step S306).

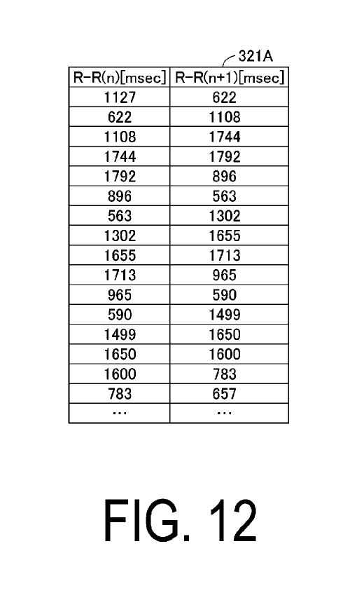

The state determination unit 512 reads inter-beat interval data from the memory 520 in a time unit necessary for determining a state, for certain time, for example, one minute, ten minutes, or an hour, and creates the correspondence relation table 321A between an inter-beat interval R−R(n) and a next inter-beat interval R−R(n+1), as illustrated in FIG. 12 (step S308).

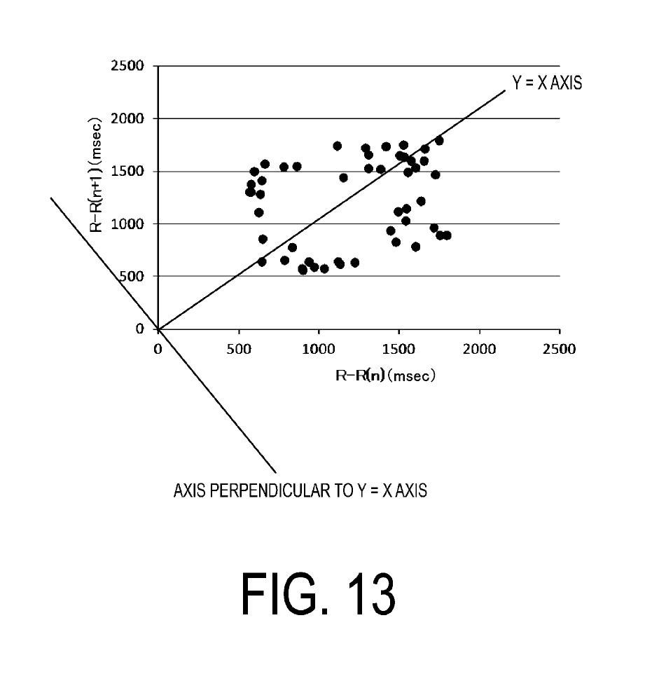

The state determination unit 512, as illustrated in FIG. 13, converts a relation table between an inter-beat interval R−R(n) and a next inter-beat interval R−R(n+1) to an axis in a Y=X direction and an axis in a direction perpendicular to the Y=X direction (step S310).

The state determination unit 512 calculates standard deviations with respect to the respective axes after axial conversion (step S312). Note that the state determination unit 512 may calculate only a standard deviation with respect to the Y=X axis, may calculate only a standard deviation with respect to the Y=−X axis, may calculate both standard deviations, or may calculate a product of both standard deviations. Additionally, the state determination unit 512, based on a calculation result, determines whether the target animal is in the measurable state or not (step S312).

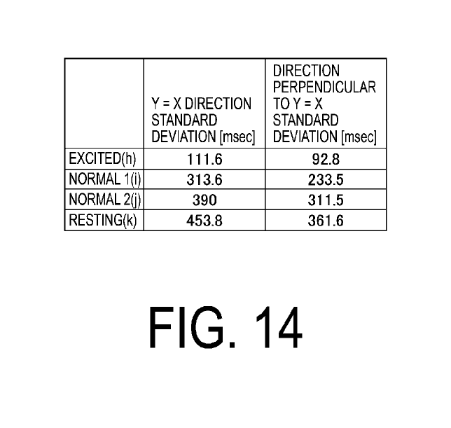

For reference, FIG. 14 is a table showing standards of standard deviations with respect to the Y=X axis, and standard deviations with respect to a Y=−X axis, per states of a dog.

In other words, in the present embodiment, the state determination unit 512, for the inter-beat intervals acquired in the signal analyzing unit 511, in a graph in which an N-th RRI is plotted on a horizontal axis, and an N+1-th RRI is plotted on a vertical axis, quantifies variation of the plots. Further, it is possible to determine whether an animal having a respiratory sinus arrthythmia such as a dog is in the measurable state or not according to magnitude and a shape of distribution of the plots.

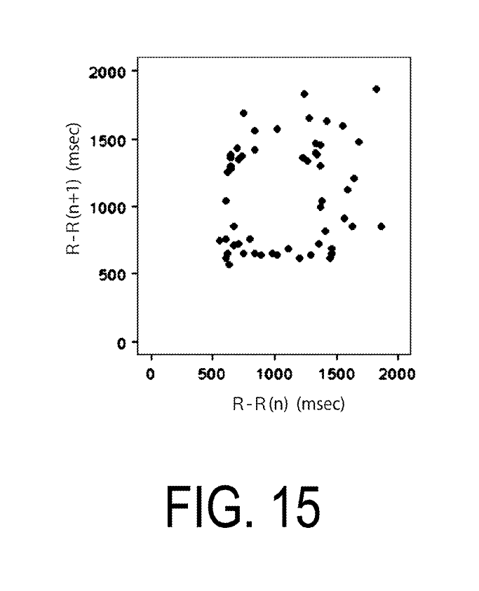

For example, a Poincaré plot of a dog in a relaxed state under a calm environment is as illustrated in FIG. 15. In the Poincaré plot in this case, the plots are distributed as a whole, and few plots exist at a center portion. In such a case, the state determination unit 512 determines the state as the “measurable state”.

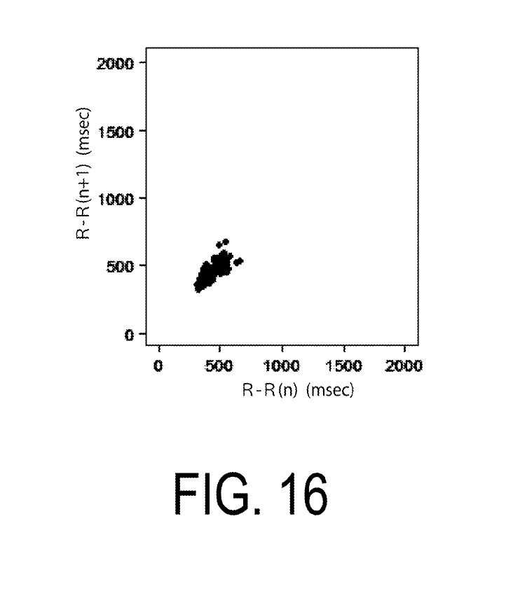

Conversely, for example, a Poincaré plot of a dog in a restless state under a noisy environment is as illustrated in FIG. 16. In the Poincaré plot in this case, the plots are crowded as a whole, and plots exist also at a center portion. In such a case, the state determination unit 512 determines the state as the “unmeasurable state”.

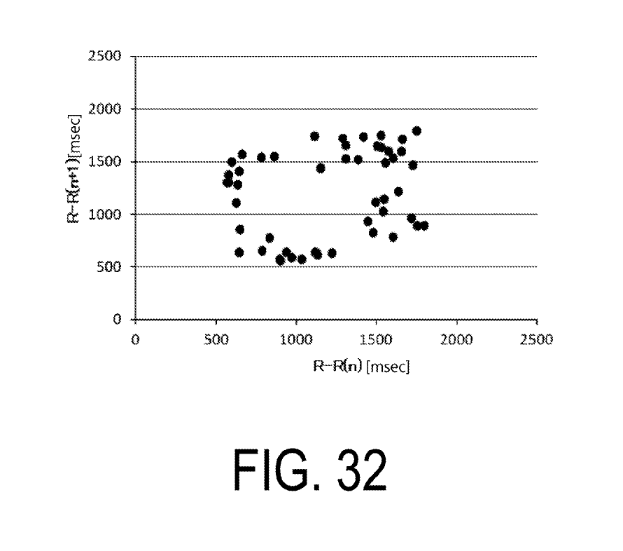

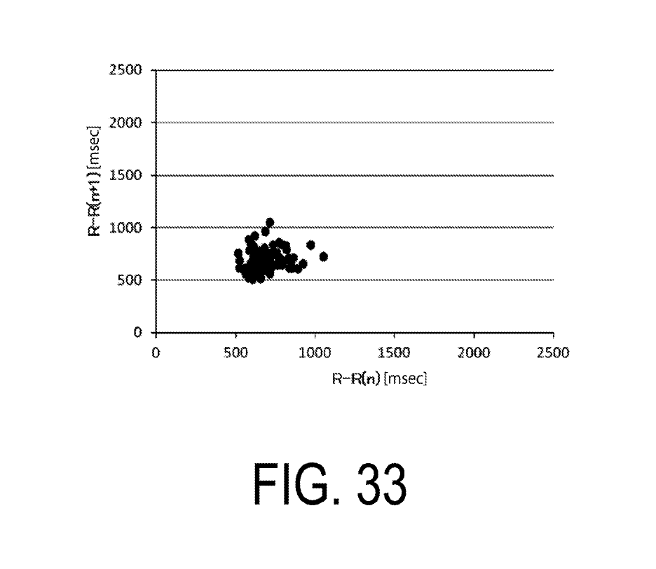

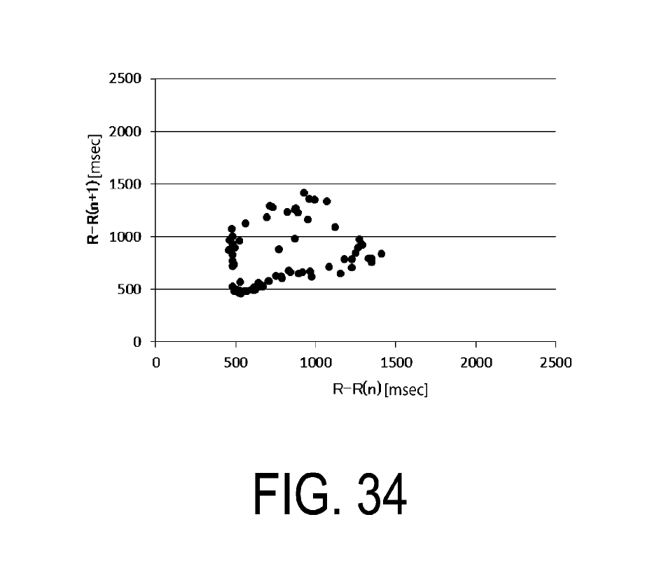

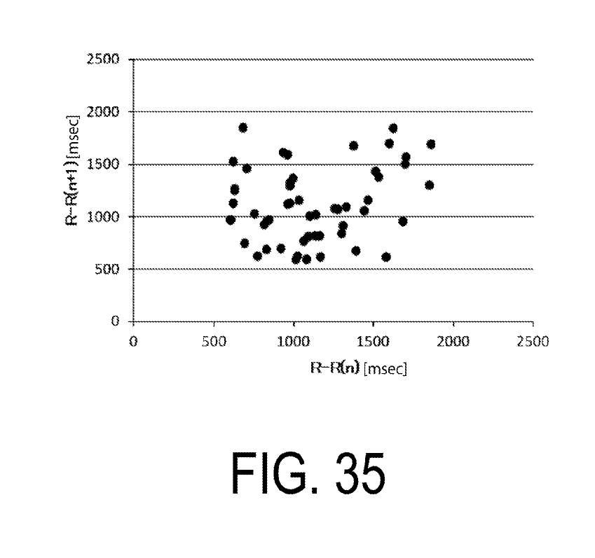

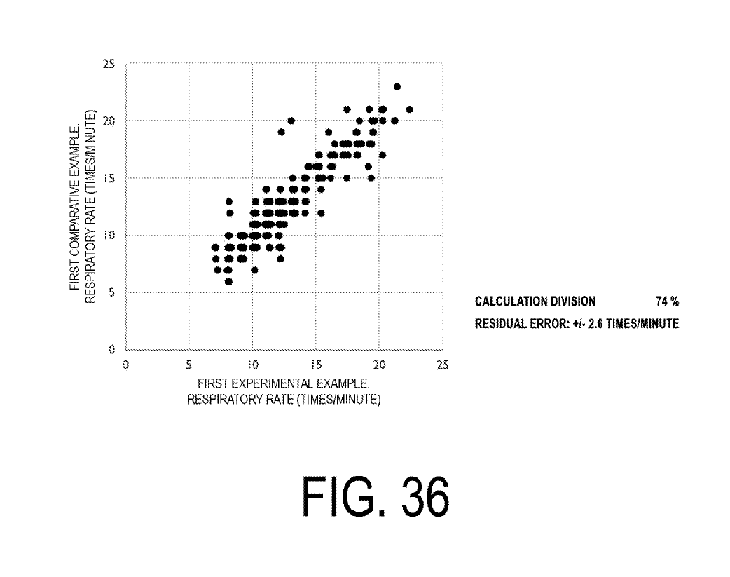

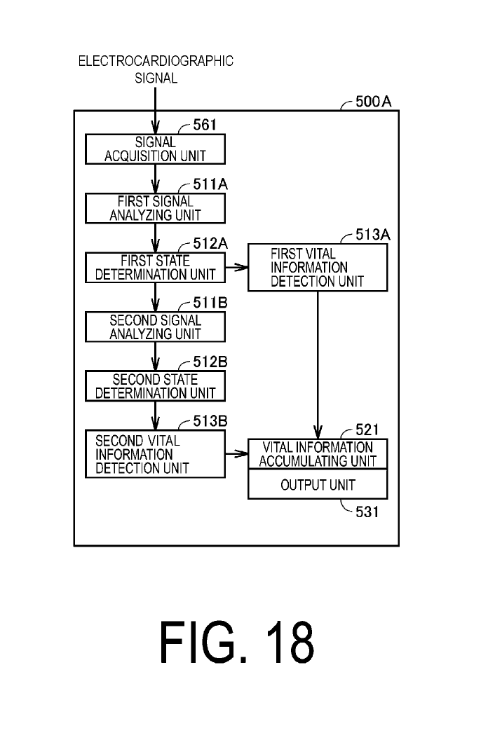

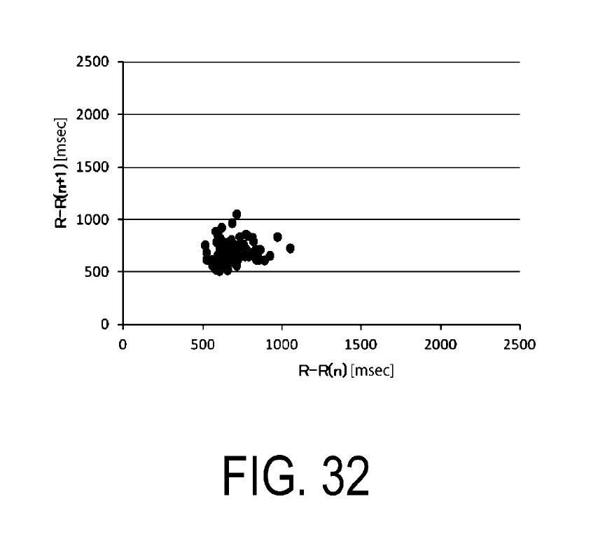

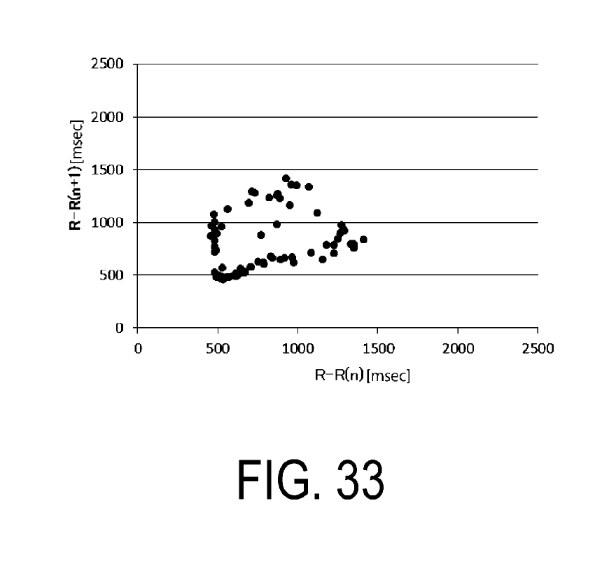

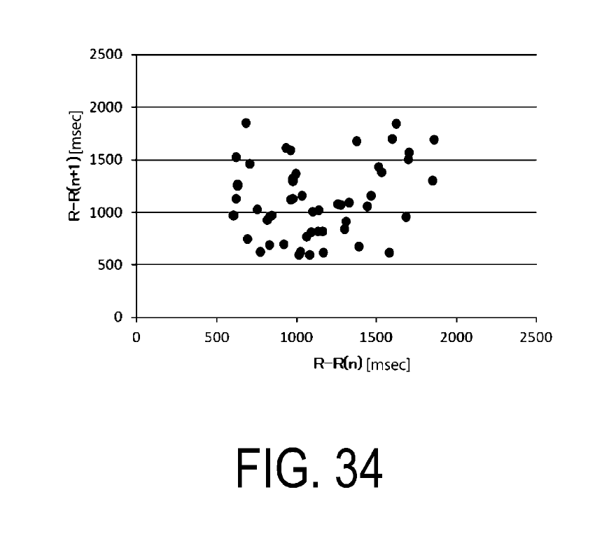

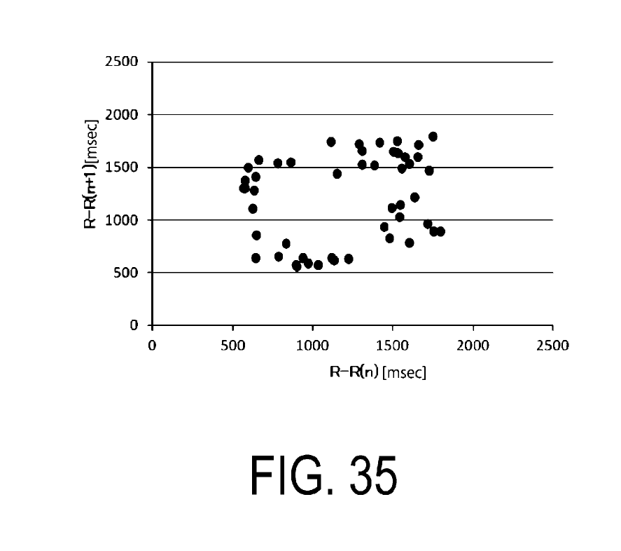

In the following, a Poincaré plot diagram will be described in more detail. FIG. 32 is a Poincaré plot diagram of a dog in an excited state. FIG. 33 is a Poincaré plot diagram of a dog with steady breathing in a normal state. FIG. 34 is a Poincaré plot diagram of a dog in a normal state. FIG. 35 is a Poincaré plot diagram of a dog in a resting state.

First, for example, in a case of an animal having a respiratory sinus arrthythmia such as a dog, in an excited state as illustrated in FIG. 32, a state occurs in which a heart rate increases (an inter-beat interval shortens), fluctuation reduces, and plots gather into a certain place.

Further, in a normal state in which breathing is stable as illustrated in FIG. 33, a heart rate is not as low as in the resting state, but an area containing few plots in a center portion of a graph (an empty hole) exists. It is considered that this shape is caused by periodic change in beat fluctuation, because heartbeat of a dog is significantly affected by breathing (respiratory sinus arrthythmia). Accordingly, it is considered that a state occurs in which the empty area exists because the breathing is stable although the beat is not slow in the relaxed state.

Further, in the normal state as illustrated in FIG. 34, a state occurs in which beat fluctuation is observed, variation expands, and plots are scattered.

Additionally, in the resting state in FIG. 35, since a dog is relaxed, the inter-beat intervals increase, and further, a shape like a circle or a rectangle, or a shape like a triangle is formed by being significantly affected by the respiratory sinus arrthythmia. Each of the shapes is a shape in which an empty portion is observed at a center portion of a Poincaré plot in the resting state.

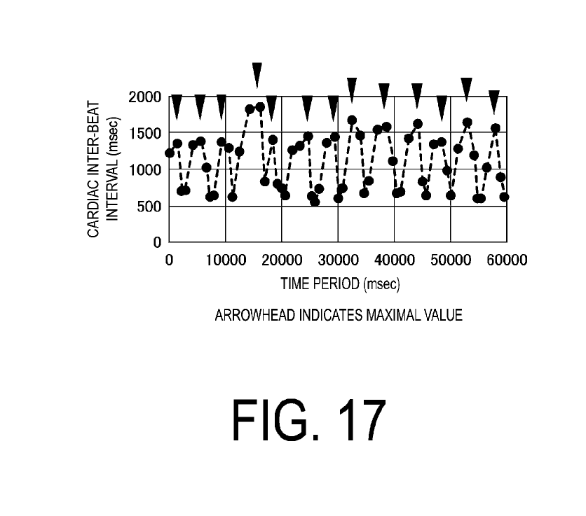

Returning to FIG. 2, the vital information detection unit 513, when the state determination unit 512 determines the state as the “measurable state”, detects vital information. In the present embodiment, the vital information detection unit 513 calculates the number of maximal (or minimal) points in time series change in inter-beat intervals as a respiratory rate, in the “measurable state”, as illustrated in FIG. 17.

In the present embodiment, when the state determination unit 512 determines the state as the “unmeasurable state”, processes in the step S308 and subsequent steps are repeated based on inter-beat intervals already acquired by the signal acquisition unit 561, for another timing. However, a processing procedure may also be adopted in which, the step S314 is executed when the state determination unit 512 sequentially executes until determination whether the state is the “unmeasurable state” or not, and only when the state is the “measurable state”.

The output unit 531 includes a display 530, a speaker 570, a communication interface 560 configured to transmit data outward, and the like. The output unit 531 displays a respiratory rate per unit time, outputs a voice, or accumulates the respiratory rate in an external data base.

### Fourth Embodiment

The first embodiment and the second embodiment utilize the power spectrum to determine whether the target animal is in the resting state or not. Additionally, in the third embodiment, based on the Poincaré plot of the inter-beat intervals, whether a target animal is in the resting state or not is determined. However, it is possible to determine whether a target animal is in the resting state or not by utilizing both of them.

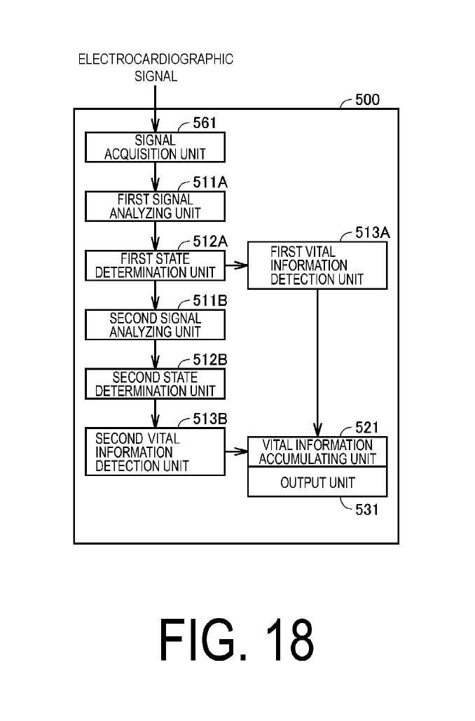

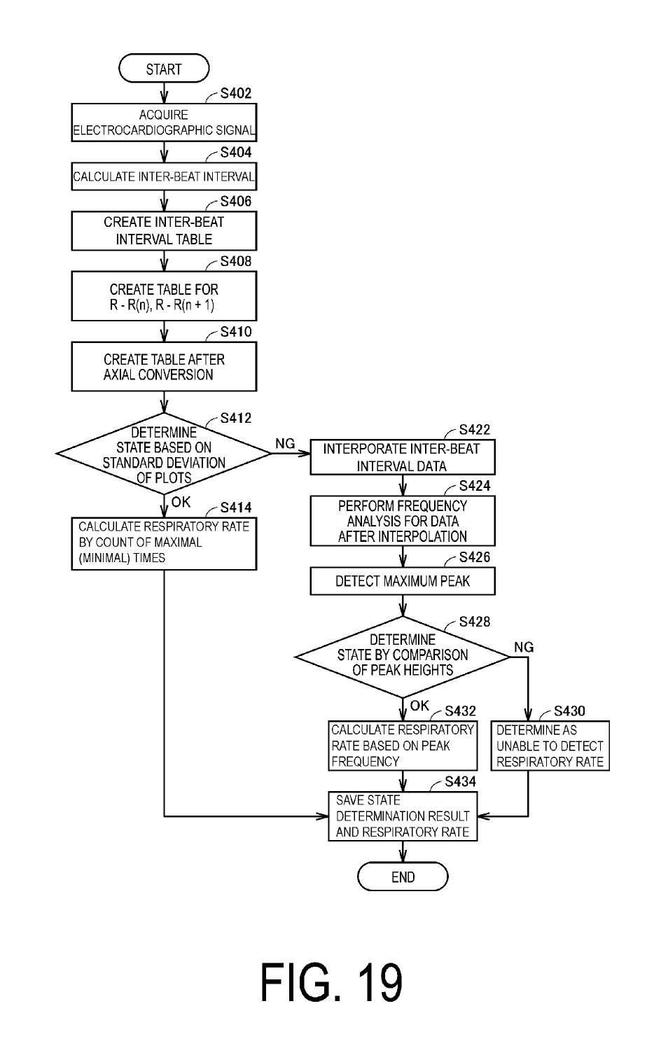

In the following, with reference to FIG. 18 and FIG. 19, a functional configuration and a processing procedure of the information processing system 1 according to the present embodiment will be described. Note that FIG. 18 is an example of a functional configuration of the information processing system 1 according to the present embodiment. FIG. 19 is a flowchart illustrating a processing procedure of the information processing system 1 according to the present embodiment.

First, a configuration of the signal processing apparatus 500A of the information processing system 1 will be described. The signal processing apparatus 500A includes the signal acquisition unit 561, a first signal analyzing unit 511A, a first state determination unit 512A, a first vital information detection unit 513A, a second signal analyzing unit 511B, a second state determination unit 512B, a second vital information detection unit 513B, a vital information accumulating unit 521, and the output unit 531.

Additionally, the signal acquisition unit 561, for example, is achieved with the communication interface 560 in FIG. 3, an electrocardiograph, a filter, an amplifier, and the like. The CPU 510 in FIG. 3 executes the programs stored in the memory 520, so that the first signal analyzing unit 511A, the first state determination unit 512A, the first vital information detection unit 513A, the second signal analyzing unit 511B, the second state determination unit 512B, and the second vital information detection unit 513B are achieved. The vital information accumulating unit 521 is achieved with the memory 520 in FIG. 3, for example. The output unit 531 is achieved with the display 530, the speaker 570, or the communication interface 560 in FIG. 3.

The signal acquisition unit 561, as illustrated in FIG. 5, for example, acquires an electrocardiographic signal at 100 Hz (step S402).

The first signal analyzing unit 511A calculates beat detection time and an inter-beat interval as illustrated in FIG. 5 from the electrocardiographic signal acquired in the signal acquisition unit 561 (step S404). The first signal analyzing unit 511A accumulates the inter-beat interval as an inter-beat interval table in the memory 520 sequentially (step S406).

The first state determination unit 512A reads inter-beat interval data from the memory 520 in time unit necessary for determining a state, for certain time, for example, one minute, ten minutes, or an hour, and creates the correspondence relation table 321A between an inter-beat interval R−R(n) and a next inter-beat interval R−R(n+1) (step S408).

The first state determination unit 512A converts the correspondence relation table 321A between an inter-beat interval R−R(n) and a next inter-beat interval R−R(n+1) to the axis in the Y=X direction and the axis in the direction perpendicular to the Y=X direction (step S410).

The first state determination unit 512A calculates standard deviations with respect to the respective axes after axial conversion (step S412). Note that the first state determination unit 512A may calculate only a standard deviation with respect to the Y=X axis, may calculate only a standard deviation with respect to the Y=−X axis, may calculate both of the standard deviations, or may calculate a product of both of the standard deviations. Additionally, the first state determination unit 512A, based on a calculation result, determines whether the target animal is in the measurable state or not (step S412).

In other words, the first state determination unit 512A, for the inter-beat intervals acquired in the first signal analyzing unit 511A, in a graph in which an N-th RRI is plotted on a horizontal axis, and an N+1-th RRI is plotted on a vertical axis, quantifies variation of the plots. Further, it is possible to determine that an animal having a respiratory sinus arrthythmia such as a dog is in the measurable state according to magnitude and a shape of distribution of the plots.

The first vital information detection unit 513A, when the state is determined as the “measurable state” (in a case of OK in the step S412) in the first state determination unit 512A, calculates the number of maximal (or minimal) points in the time series change of the inter-beat intervals acquired in the signal analyzing unit 511 as a respiratory rate per unit time, as illustrated in FIG. 17.

Further, the vital information accumulating unit 521 accumulates the respiratory rate, and the output unit 531 makes the display 530, the speaker 570, the communication interface 560 configured to transmit data outward, output the respiratory rate (step S434).

On the other hand, when the state is not determined as the “measurable state” in the first state determination unit 512A (in a case of not good (NG) in the step S412), the second signal analyzing unit 511B, for example, as illustrated in FIG. 6, mathematically interpolates (e.g., spline interpolation) relation between beat detection time and an inter-beat interval for one minute (step S422). More specifically, the second signal analyzing unit 511B detects a peak signal (R wave) of electrocardiographic signals by a method such as threshold detection, and calculates respective periods (times) between the peaks of the electrocardiographic signals. As a calculation method of the inter-beat interval, in addition to the above method, derivation of a cycle using an autocorrelation function, a method using a square wave correlation trigger, or the like, may be adopted.

Additionally, the second signal analyzing unit 511B, as illustrated in FIG. 7, performs frequency analysis by an acquired function (step S424).

The second state determination unit 512B, in power spectral distribution as in FIG. 7 acquired by the frequency analysis in the second signal analyzing unit 511B, and within an arbitrary frequency range (e.g., from 0.05 to 0.5 Hz), finds out a peak for which a maximum peak of power spectrum is maximum (step S426).

The second state determination unit 512B, depending on whether a ratio of the maximum peak compared to a second largest peak is equal to or larger than an arbitrary threshold value (e.g., three times) or not, determines whether the state is the “measurable state” or not (step S428).

When the state is determined as the “measurable state” in the second state determination unit 512B (in a case of OK in the step S428), the second vital information detection unit 513B, with a maximum peak in an arbitrary frequency range (e.g., from 0.05 to 0.5 Hz) in the frequency analysis performed in the second state determination unit 512B being a breathing frequency, calculates a respiratory rate per unit time by calculating an inverse of the frequency (step S432).

Further, the vital information accumulating unit 521 accumulates the respiratory rate, and the output unit 531 makes the display 530, the speaker 570, the communication interface 560 configured to transmit data outward, output the respiratory rate (step S434).

Note that when the state is not determined as the “measurable state” in the second state determination unit 512B (in a case of NG in the step S428), the output unit 531 allows an error message indicating “unable to detect a respiratory rate” to be outputted via the display 530, the speaker 570, the communication interface 560 configured to transmit data outward, and the like (step S430). However, the CPU 510, when the state is not determined as the “measurable state” (in a case of NG in the step S428), may repeat the processes in the step S408 and the subsequent steps, for another timing.

In the present embodiment, since whether measurement is possible or not is determined in advance based on the Poincaré plot, it is possible to reduce a calculation amount compared to determination using a histogram. However, an embodiment may be adopted in which the determination using a histogram is performed in advance, and when the measurement is determined as impossible, whether the measurement is possible or not is determined based on the Poincaré plot.

### Fifth Embodiment

In the first to the fourth embodiments, the electrode 400 configured to acquire an electrocardiographic signal and attached to the chest of a dog is utilized. However, an electrode attachment location is not limited to the embodiment.

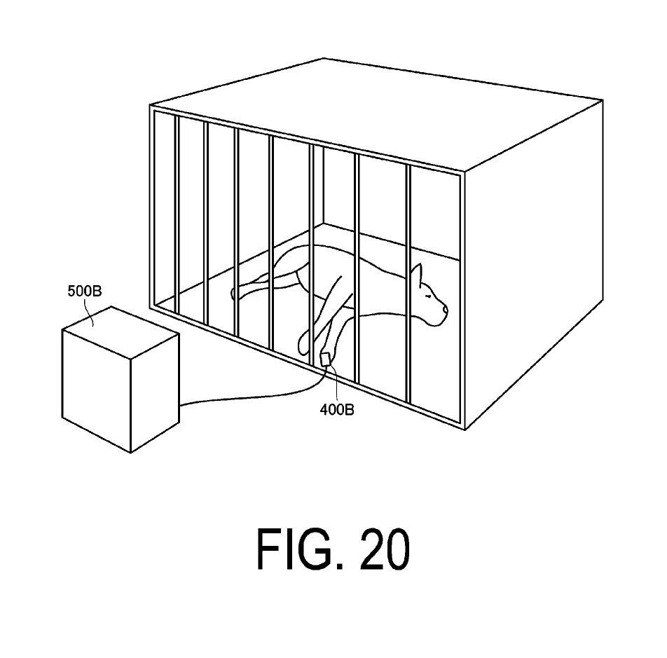

For example, as illustrated in FIG. 20, the electrode 400B configured to measure an electrocardiographic signal may be attached on a sole or the like, and the electrocardiographic signal may be transmitted to a vital information monitor 500B. Note that subsequent signal analysis, state determination, and vital information detection are similar to those in other embodiments, and thus descriptions will not be repeated here. More specifically, the vital information monitor 500B may be installed with a function of the signal processing apparatus 500 according to the first to the fourth embodiments, or the vital information monitor 500B may provide electrocardiac data to another device installed with the function of the signal processing apparatus 500 according to the first to the fourth embodiments via a wired or wireless network.

### Sixth Embodiment

In the first to the fourth embodiments, the electrode 400 configured to acquire an electrocardiographic signal and attached to a chest of a dog is utilized. However, the present invention is not limited to the embodiment.

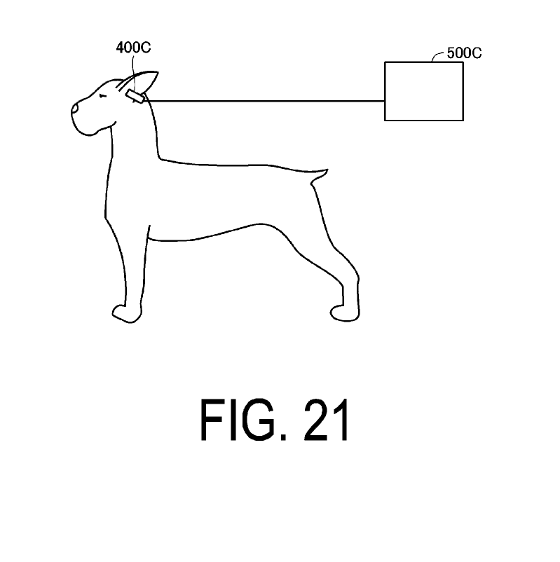

For example, as illustrated in FIG. 21, a configuration may be adopted in which an photoelectric pulse wave type sphygmograph 400C acquires a pulse wave signal, and the pulse wave signal is transmitted to a vital information monitor 500C. In this case, a portion on which a pulse wave is measured is preferably a portion on which skin is exposed including a tongue, an ear, or the like. Note that subsequent signal analysis, state determination, and vital information detection are similar to those in the first to the fourth embodiments, and thus descriptions will not be repeated here. More specifically, the vital information monitor 500C may be installed with a function of the signal processing apparatus 500 according to the first to the fourth embodiments, or the vital information monitor 500C may provide electrocardiac data to another device installed with the function of the signal processing apparatus 500 according to the first to the fourth embodiments via a wired or wireless network.

### Seventh Embodiment

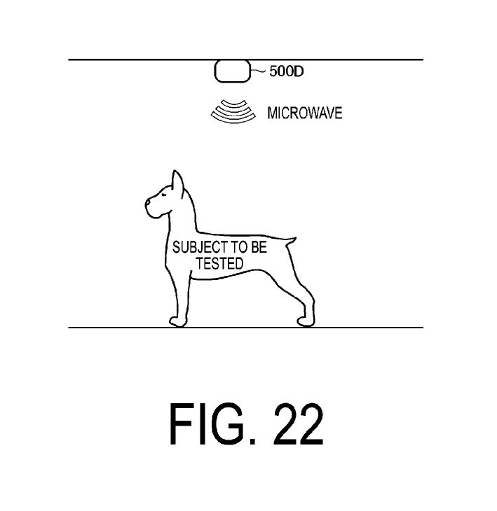

Alternatively, as illustrated in FIG. 22, a pulse may be detected by utilizing a pulse wave acquisition sensor such as a microwave Doppler sensor. For example, an embodiment is conceivable in which a microwave transmission device 500D is installed on a ceiling or the like, and a pulse wave is acquired from an animal such as a dog without contact. More specifically, signal processing is performed in which only heartbeat is detected from raw data of a microwave Doppler sensor. Subsequent signal analysis, state determination, and vital information detection are similar to those in the first to the fourth embodiments, and thus descriptions will not be repeated here.

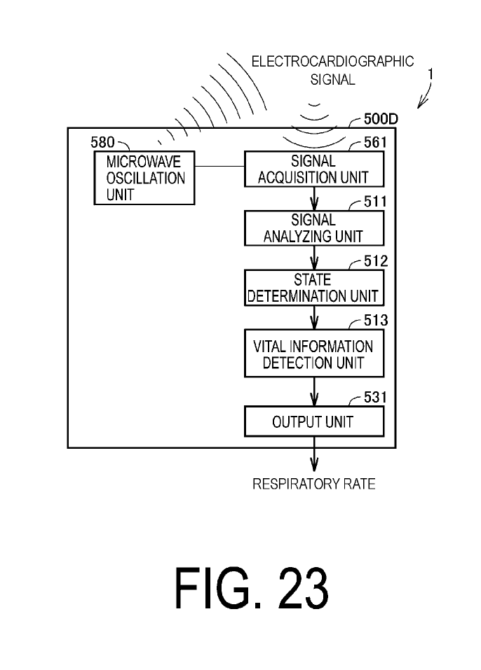

More specifically, as illustrated in FIG. 23, the microwave transmission device 500D may be installed with the function of the signal processing apparatus 500 according to the first to the fourth embodiments, and a microwave oscillation unit 580. Note that in this case, the signal acquisition unit 561 needs to be capable of detecting a reflection wave of a microwave.

Naturally, the microwave transmission device 500D may be separated from another device installed with the function of the signal processing apparatus 500 according to the first to the fourth embodiments.

The present experimental example enables measurement without contact, and has an effect of reducing a load for a subject to be tested.

### Eighth Embodiment

In the information processing system 1 according to the first to the seventh embodiments, based on the electrocardiographic signal from the electrode 400, the signal processing apparatus 500 determines whether a state is the measurable state or not, and calculates or outputs a respiratory rate. However, all or part of a role of any of the above devices may be played by other devices, or shared among a plurality of devices. Conversely, all or part of roles of the plurality of devices may be played by one device, or played by another device.

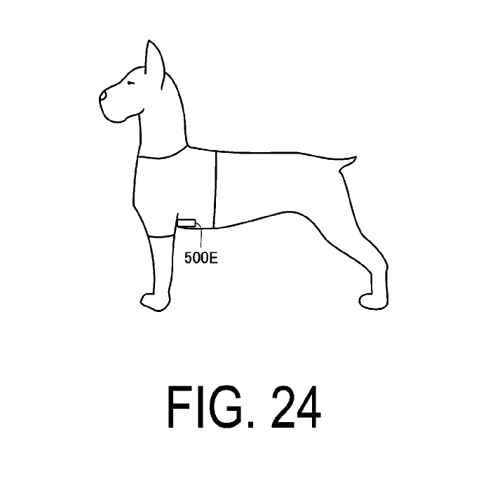

For example, as illustrated in FIG. 24, a signal processing apparatus 500E may be mounted with a sensor such as the electrode 400 integrally.

### Ninth Embodiment

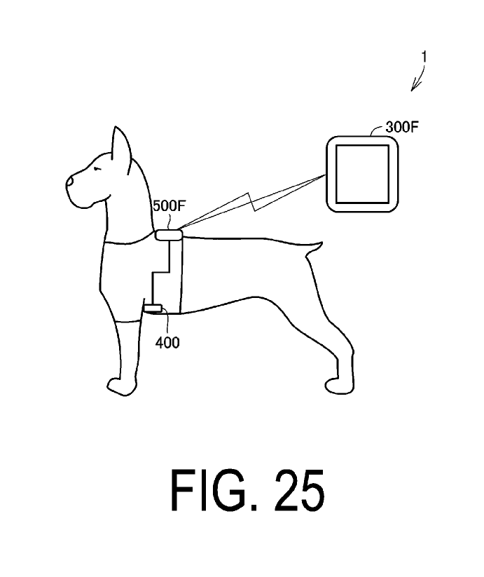

Alternatively, as illustrated in FIG. 25, part of a role of the signal processing apparatus 500 may be played by a communication terminal 300F capable of communicating with a signal processing apparatus 500F.

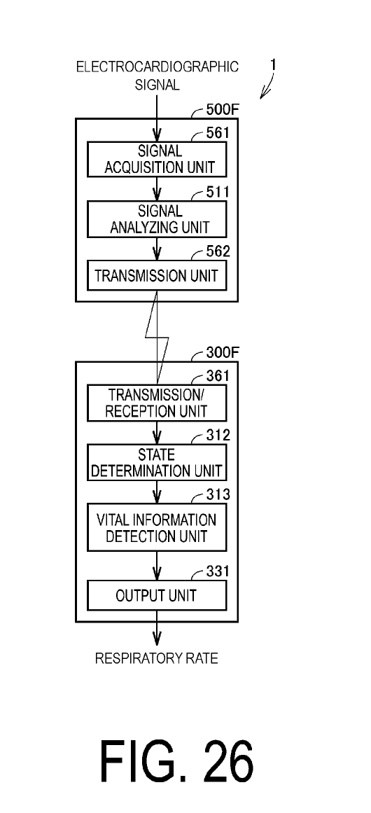

More specifically, as illustrated in FIG. 26, the signal processing apparatus 500F according to the present embodiment, mainly includes functions of the signal acquisition unit 561, the signal analyzing unit 511, and a transmission unit 562. Note that the signal acquisition unit 561 includes an electrocardiograph, the communication interface 560 in FIG. 3, a filter, an amplifier, and the like. The transmission unit 562 is achieved with the communication interface 560 illustrated in FIG. 3 or the like. The CPU 510 illustrated in FIG. 3 executes the programs stored in the memory 520, so that the signal analyzing unit 511 is achieved.

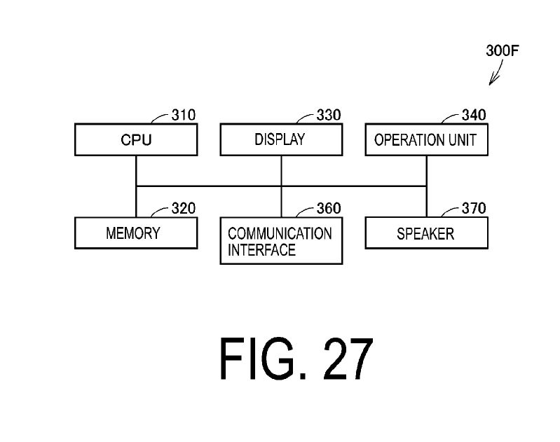

Further, as illustrated in FIG. 26, the communication terminal 300F includes a transmission/reception unit 361, the state determination unit 312, a vital information detection unit 313, and an output unit 331. Note that the transmission/reception unit 361 is achieved with a communication interface 360 illustrated in FIG. 27. A CPU 310 illustrated in FIG. 27 executes programs stored in a memory 320, so that the state determination unit 312 and the vital information detection unit 313 are achieved. The output unit 331 is achieved with the display 330, a speaker 370, the communication interface 360 configured to transmit data outward, and the like.

First, the signal acquisition unit 561, as illustrated in FIG. 5, for example, acquires an electrocardiographic signal at 100 Hz. The signal analyzing unit 511 calculates beat detection time and an inter-beat interval as illustrated in FIG. 5 from the electrocardiographic signal acquired in the signal acquisition unit 561.

Additionally, the signal analyzing unit 511, for example, as illustrated in FIG. 6, mathematically interpolates (e.g., spline interpolation) the relation between beat detection time and an inter-beat interval for one minute. More specifically, the signal analyzing unit 511 detects a peak signal (R wave) among electrocardiographic signals by a method such as threshold detection, and calculates respective periods (times) between the peaks of the electrocardiographic signals. As a calculation method of the inter-beat interval, in addition to the above method, derivation of a cycle using an autocorrelation function, a method using a square wave correlation trigger, or the like, may be adopted.

Additionally, the signal analyzing unit 511, as illustrated in FIG. 7, performs frequency analysis by an acquired function. The transmission unit 562 transmits a result of the frequency analysis to the communication terminal 300F.

The transmission/reception unit 361 of the communication terminal 300F receives data from the signal processing apparatus 500. The state determination unit 312, in power spectral distribution as in FIG. 7 acquired by the frequency analysis in the signal analyzing unit 511, and within an arbitrary frequency range (e.g., from 0.05 to 0.5 Hz), finds out a peak for which a maximum peak of power spectrum is maximum. The state determination unit 312, when a ratio of the maximum peak compared to a second largest peak is equal to or larger than an arbitrary threshold value (e.g., three times), determines the state as the “measurable state”.

The vital information detection unit 313, when the state determination unit 312 determines the state as the “measurable state”, detects vital information. The vital information detection unit 313, with a maximum peak in an arbitrary frequency range (e.g., from 0.05 to 0.5 Hz) in the frequency analysis performed in the state determination unit 312 being a breathing frequency, calculates a respiratory rate by calculating an inverse.

The output unit 331 displays a respiratory rate per unit time, outputs a voice, or accumulates the respiratory rate in a database, via the display 330, the speaker 370, and the communication interface 360 configured to transmit data outward.

Note that role sharing between the signal processing apparatus 500F and the communication terminal 300F is not limited to that described above, and part of a function of the signal analyzing unit 511 may be played by the communication terminal 300F, or part of functions of the state determination unit 312, the vital information detection unit 313, and the output unit 331 may be played by the signal processing apparatus 500F.

### Tenth Embodiment

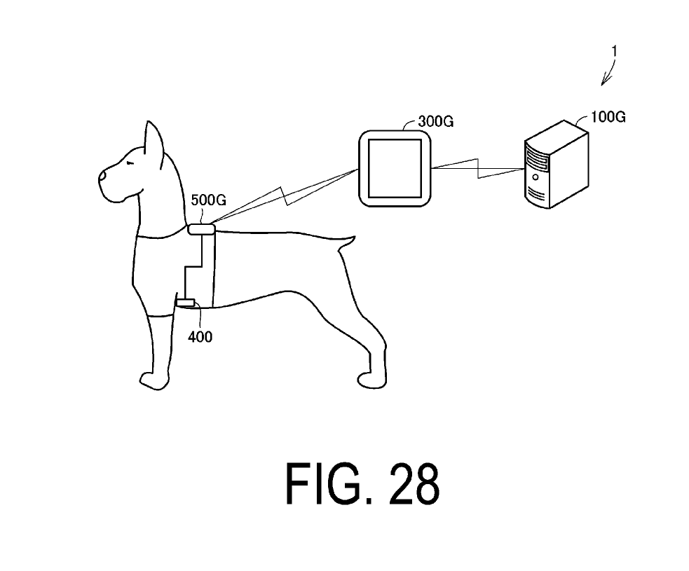

Alternatively, as illustrated in FIG. 28, part of a role of the signal processing apparatus 500F may be played by the server 100G capable of communicating with the communication terminal 300G capable of communicating with the signal processing apparatus 500G.

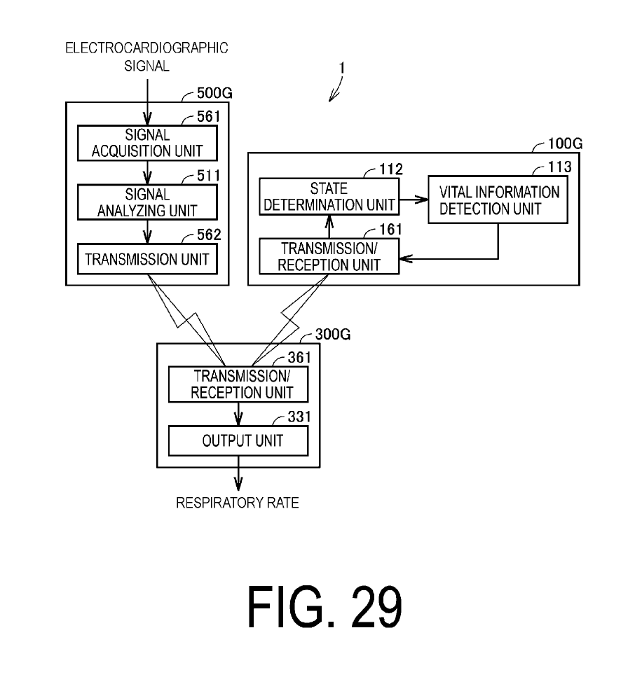

More specifically, as illustrated in FIG. 29, the signal processing apparatus 500G mainly includes functions of the signal acquisition unit 561, the signal analyzing unit 511, and the transmission unit 562. Note that the signal acquisition unit 561 includes an electrocardiograph, the communication interface 560 in FIG. 3, a filter, an amplifier, and the like. The transmission unit 562 is achieved with the communication interface 560 illustrated in FIG. 3 or the like. The CPU 510 illustrated in FIG. 3 executes the programs stored in the memory 520, so that the signal analyzing unit 511 is achieved.

Further, as illustrated in FIG. 29, the communication terminal 300G includes the transmission/reception unit 361 and the output unit 331. Note that the transmission/reception unit 361 is achieved with a communication interface 360 illustrated in FIG. 27. The output unit 331 is achieved with the display 330, the speaker 370, and the like.

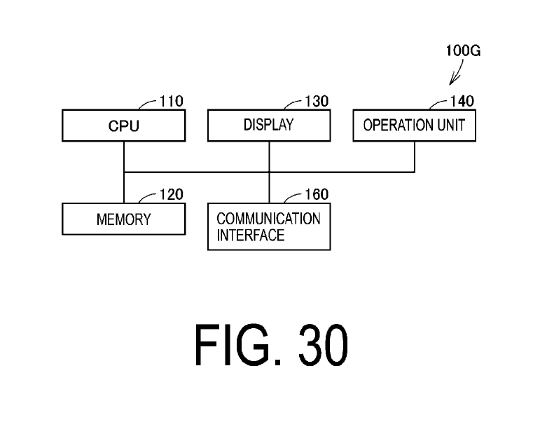

Further, as illustrated in FIG. 29, the server 100G includes a transmission/reception unit 161, a state determination unit 112, and a vital information detection unit 113. Note that the transmission/reception unit 161 is achieved with a communication interface 160 illustrated in FIG. 30. A CPU 110 illustrated in FIG. 30 executes programs stored in a memory 120, so that the state determination unit 112 and the vital information detection unit 113 are achieved.

First, the signal acquisition unit 561, as illustrated in FIG. 5, for example, acquires an electrocardiographic signal at 100 Hz. The signal analyzing unit 511 calculates beat detection time and an inter-beat interval as illustrated in FIG. 5 from the electrocardiographic signal acquired in the signal acquisition unit 561.

Additionally, the signal analyzing unit 511, for example, as illustrated in FIG. 6, mathematically interpolates (e.g., spline interpolation) relation between beat detection time and an inter-beat interval for one minute. More specifically, the signal analyzing unit 511 detects a peak signal (R wave) among electrocardiographic signals by a method such as threshold detection, and calculates respective periods (times) between the peaks of the electrocardiographic signals. As a calculation method of the inter-beat interval, in addition to the above method, derivation of a cycle using an autocorrelation function, a method using a square wave correlation trigger, or the like, may be adopted.

Additionally, the signal analyzing unit 511, as illustrated in FIG. 7, performs frequency analysis by an acquired function. The transmission unit 562 transmits a result of the frequency analysis to the communication terminal 300G.

The transmission/reception unit 361 of the communication terminal 300G receives data from the signal processing apparatus 500G and transmits the data to the server 100G.

The transmission/reception unit 161 of the server 100G receives data from the communication terminal 300G. The state determination unit 112, in power spectral distribution as in FIG. 7 acquired by the frequency analysis in the signal analyzing unit 511, and within an arbitrary frequency range (e.g., from 0.05 to 0.5 Hz), finds out a peak for which a maximum peak of power spectrum is maximum. The state determination unit 112, when a ratio of the maximum peak compared to a second largest peak is equal to or larger than an arbitrary threshold value (e.g., three times), determines the state as a “measurable state”.

The vital information detection unit 113, when the state determination unit 112 determines the state as the “measurable state”, detects vital information. The vital information detection unit 113, with a maximum peak in an arbitrary frequency range (e.g., from 0.05 to 0.5 Hz) in the frequency analysis performed in the state determination unit 112 being a breathing frequency, calculates a respiratory rate by calculating an inverse.

The transmission/reception unit 161 of the server 100G transmits data such as a respiratory rate to the communication terminal 300G. The transmission/reception unit 361 of the communication terminal 300G receives data from the server 100G. Additionally, the output unit 331 outputs a respiratory rate per unit time, via the display 330, the speaker 370, the communication interface 360 configured to transmit data outward, and the like.

Note that role sharing among the signal processing apparatus 500G, the communication terminal 300G, and the server 100G is not limited to that described above, for example, and part of a function of the signal analyzing unit 511 may be played by the communication terminal 300G or the server 100G, or part of functions of the state determination unit 312, and the vital information detection unit 313 may be played by the communication terminal 300G or the signal processing apparatus 500G. Additionally, part of a function of the output unit 331 may be played by another smart phone, tablet, or personal computer capable of communicating with the server 100G, the communication terminal 300G, or the signal processing apparatus 500G.

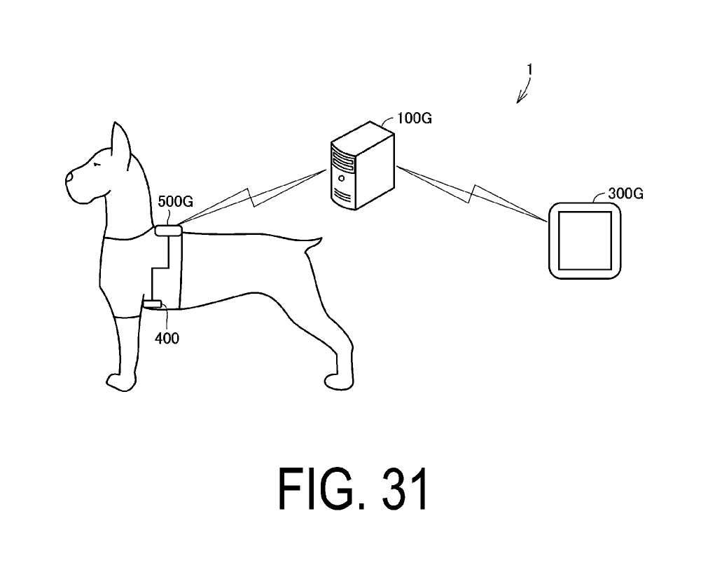

Note that naturally, as illustrated in FIG. 31, the signal processing apparatus 500G may be capable of communicating with a server 100G via a router or the Internet, or the communication terminal 300G may be capable of communicating with the server 100G via the Internet, or a carrier network.

### Other Application Examples

It is needless to say that an embodiment of the present invention is applicable to a case achieved by supplying programs to a system or a device. Additionally, it is also possible to benefit from the effect of an embodiment of the present invention by supplying a storage medium (or a memory) storing programs expressed by software for achieving the embodiment of the present invention to a system or a device, and by a computer of the system or the device (or a CPU or an MPU) reading and executing program codes stored in the storage medium.

In this case, the program codes themselves read from the storage medium achieve functions of the above-described embodiment, and the storage medium storing the program codes constitutes an embodiment of the present invention.

Additionally, it is needless to say not only that the functions of the above-described embodiment are enabled by a computer reading and executing the program codes, but also that a case is also included, in which, based on instructions of the program codes, an Operating System (OS) running on the computer or the like executes part or all of actual processes, and the functions of the above-described embodiment are enabled by the processes.

Further, it is needless to say that a case is also included, in which, after the program codes read from the storage medium are written to another storage medium provided in a function expansion board inserted into the computer or a function expansion unit connected to the computer, based on instructions of the program codes, a CPU or the like provided in the function expansion board or the function expansion unit executes part or all of actual processes, and functions of the above-described embodiment are enabled by the processes.

### Experimental Example and Comparative Example

Here, in order to examine how precisely the information processing system of the first embodiment is capable of measuring a respiratory rate of a subject to be tested, the following first experimental example and first comparative example were prepared.

In the first experimental example, each of four beagle dogs (the minimum body weight is 9.1 kg, the maximum body weight is 13.3 kg, the youngest age is 11 months, the oldest age is 19 months) as a subject to be tested was fitted with a vest-shaped measurement device, and a respiratory rate of the above subject to be tested was measured using the information processing system of the present embodiment. During the measurement of the respiratory rate, the subject to be tested was allowed to move inside a cage with 60×72×55 cm. Additionally, the subject to be tested was fitted with the measurement device, 30 minutes before start of the measurement, and the measurement started after sufficiently accustoming the subject to be tested to an experimental environment. Total measurement time for the four subjects to be tested was 526 minutes.

In the first comparative example, a nasal cavity of the subject to be tested identical to that in a first experimental example was fitted with a thermopile (MLX90613DAA, Melexis Technology, NV), and in parallel to the first experimental example, a respiratory rate of the above-described subject to be tested was measured based on temperature change in exhaled breath and inhaled breath detected by the thermopile.

FIG. 36 is a diagram made by plotting respiratory rates per minute for four subjects to be tested measured in each of the first experimental example and the first comparative example. A horizontal axis in FIG. 36 indicates the respiratory rate (times/minute) measured in the first experimental example, and a vertical axis in FIG. 36 indicates the respiratory rate (times/minute) measured in the first comparative example.

Of 526 minutes of the total measurement time for the four subjects to be tested in the first experimental example, total time for which the respiratory rates for the four subjects to be tested were determined to be in the “measurable state” was 388 minutes (74% of total measurement duration). A residual error between the first experimental example and the first comparative example in this “measurable state” was +/−2.6 times/minute. This residual error showed that the respiratory rate measurement method by the information processing system 1 disclosed in the first embodiment has sufficient precision.

The embodiments disclosed here are to be understood as being in all ways exemplary and in no ways limiting. The scope of the present invention is defined not by the foregoing descriptions but by the appended claims, and is intended to include all changes equivalent in meaning and scope to the claims.

### REFERENCE SIGNS LIST

1: Information processing system
100G: Computer (server)
110: Processor (CPU)
112: State determination unit
113: Vital information detection unit
120: Memory
130: Display
140: Operation unit
160: Communication interface (output device)
161: Transmission/reception unit
300F: Computer (communication terminal)
300G: Computer (communication terminal)
310: Processor (CPU)
312: State determination unit
313: Vital information detection unit
320: Memory
321A: Correspondence relation table
330: Display (output device)
331: Output unit
340: Operation unit
360: Interface (output device)
361: Transmission/reception unit
370: Speaker
400: Sensor (electrode)
400B: Electrode
400C: Sphygmograph
500: Computer (signal processing apparatus)
500B: Vital information monitor
500C: Vital information monitor
500D: Microwave transmission device
500E: Signal processing apparatus
500F: Signal processing apparatus
500G: Signal processing apparatus
510: Processor (CPU)
511: Signal analyzing unit
511A: First signal analyzing unit
511B: Second signal analyzing unit
512: State determination unit
512A: First state determination unit
512B: Second state determination unit
513: Vital information detection unit
513A: First vital information detection unit
513B: Second vital information detection unit
520: Memory
521: Vital information accumulating unit
530: Display (output device)
531: Output unit
540: Operation unit
560: Interface (output device)
561: Signal acquisition unit
562: Transmission unit
570: Speaker
580: Microwave oscillation unit

1: A computer, comprising:
an interface configured to acquire data indicating a pulse or heartbeat of an animal; and
a processor configured to determine whether a prescribed condition is satisfied or not based on the data indicating a pulse or heartbeat of an animal, and to calculate a respiratory rate in a period of time during which the prescribed condition is satisfied.

2: The computer according to claim 1,
wherein the processor is configured to calculate the respiratory rate from the data of a pulse or heartbeat of an animal.

3: The computer according to claim 1 or 2,
wherein the processor is configured to process the data of a pulse or heartbeat of an animal sequentially, and to calculate a respiratory rate from the data of a pulse or heartbeat of an animal in a period of time during which the prescribed condition is satisfied.

4: The computer according to claim 1,
wherein the processor is configured to calculate an inter-beat interval from the data of a pulse or heartbeat of the animal, and to calculate the respiratory rate based on the inter-beat interval.

5: The computer according to claim 4,
wherein the processor is configured to create a power spectrum of the inter-beat interval, to determine whether the prescribed condition is satisfied or not based on the power spectrum, and to acquire the respiratory rate based on the power spectrum.

6: The computer according to claim 4,
wherein the processor is configured to determine whether the prescribed condition is satisfied or not based on a Poincaré plot of the inter-beat interval.

7: The computer according to claim 1,
wherein a target animal has a respiratory sinus arrthythmia.

8: A method of acquiring a respiratory rate of an animal, the method performed on a computer including a processor, the method comprising:
acquiring data indicating a pulse or heartbeat of an animal;
determining whether a prescribed condition is satisfied or not based on the data indicating a pulse or heartbeat of an animal; and
acquiring a respiratory rate in a period of time during which the prescribed condition is satisfied.

9: An information processing system, comprising:
an output device;
a sensor configured to detect a beat of an animal; and
a computer configured to determine whether a prescribed condition is satisfied or not based on data indicating a pulse or heartbeat of the animal from the sensor, to calculate a respiratory rate in a period of time during which the prescribed condition is satisfied, and to cause the output device to output.
